(19) United States
(12) Patent Application Publication
(10) Pub. No.: US 2008/0275015 A1
(43) Pub. Date: Nov. 6, 2008

(54) Formulation and Method for Treating Interstitial Cystitis and Related Bladder Conditions

(76) Inventor: Jeffrey A. Potter, Wadsworth, OH (US)

Correspondence Address: BUCKINGHAM, DOOLITTLE & BURROUGHS, LLP, 3800 EMBASSY PARKWAY, SUITE 300, AKRON, OH, 44333-8332, US

(21) Appl. No.: 12/111,234

(22) Filed: Apr. 29, 2008

Related U.S. Application Data
(60) Provisional application No. 60/915,126, filed on May 1, 2007.

(51) Int. Cl. A61K 31/56; A61K 31/495; A61K 31/195; A61K 31/198; A61P 13/10

(52) U.S. Cl. 514/180; 514/255.04; 514/561; 514/565

(57) ABSTRACT

The invention of this application relates to a formulation and method of using the formulation for the treatment of interstitial cystitis and other similar conditions of the bladder. The formulation of this invention is a highly specialized, compounded pharmaceutical that contains a combination of numerous therapeutically different medications in a unique delivery vehicle and system. The individual components of the formulation work synergistically to restore and repair each of the different issues associated with interstitial cystitis. The individual components of the formulation exist in an aqueous vehicle to facilitate drug contact with the bladder wall. The formulation is instilled directly into the bladder, commonly in a physician's office.

### TECHNICAL FIELD

The invention of this application relates to a formulation and method of using the formulation for the treatment of interstitial cystitis and other similar conditions of the bladder.

### BACKGROUND OF THE INVENTION

Interstitial cystitis, also known as painful bladder syndrome, is a chronic, idiopathic condition of the bladder characterized by pelvic pain as well as excessive urinary urgency and frequency. This condition is often debilitating, interfering with daily activities, social functioning, and sexual activity. The disease afflicts upwards of 700,000 women, who comprise roughly 90% of the population affected by the condition. The etiology and pathophysiology of interstitial cystitis is largely unknown. It is often described in the medical literature as a neuron-immuno-endocrine disorder, meaning that the nervous, immune and hormonal systems of the body may all be involved in interstitial cystitis.

Pelvic pain is the most prominent symptom of interstitial cystitis. It is often relieved upon urination but returns as the bladder fills. However, urinary urgency is not usually relieved with urination. Urinary frequency averages 16 times per day but can be as high as 60 times per day in some patients. This frequency can contribute to nocturia (urination at night), but usually does not include incontinence. Another symptom experienced by many patients is dyspareunia (pain with sexual intercourse).

The clinical course of this disease usually involves flare-ups followed by periods of remission. In women, symptoms may worsen during ovulation or during the premenstrual period. Symptoms may be exacerbated by acidic foods such as coffee, carbonated beverages, other caffeinated products, alcohol, citrus fruits, spicy foods, and tomatoes. Some patients are also sensitive to foods containing high levels of arylalkylamines (i.e. tryptophan, tyrosine, tyramine, phenylalanine) such as beer, cheese, bananas, chocolate, wine, and yoghurt.

This condition is extremely detrimental to one's quality of life and may often be misdiagnosed or go undiagnosed. It is important to be aware of the symptoms surrounding this syndrome and to educate oneself about options in therapy. Common characteristics of interstitial cystitis include, but are not limited to: the presence of pelvic, suprapubic, perineal, urethral or bladder pain; the presence of urinary urgency or frequency; pain with intercourse; bladder capacity of less than 350 mL; and negative urine cultures. Approximately 90% of affected patients have non-ulcerous interstitial cystitis, and approximately 10% are affected with the more serious, ulcerous form of interstitial cystitis in which Hunner's ulcers are found upon cytoscopic examination.

Symptoms of interstitial cystitis may present similarly to those of urinary tract infection, but this condition is not infectious in nature—urine cultures are negative for bacteria. While the cause of interstitial cystitis is not fully understood or known, one theory postulates that the chronic bladder inflammation and irritation is caused by a defect in the protective mucous layer of the bladder. This inner lining of the bladder has lower rates of glycosaminoglycan (GAG) excretion in patients with interstitial cystitis. Glycosaminoglycan protects the bladder from damage by maintaining a stable layer of water and by preventing invasion by bacteria and irritation from substances in the urine. In interstitial cystitis, the flawed defensive layer allows irritating substances to seep through the urothelium that may lead to injury. The body responds to this injury by releasing inflammatory mediators and histamine, resulting in the symptoms associated with the disease.

Currently, the Prior Art provides no cure for interstitial cystitis. The goal of Prior Art treatments and therapies is to alleviate the symptoms of interstitial cystitis, rather than curing the condition. Most patients with interstitial cystitis are able to identify foods and other factors that exacerbate their symptoms Lifestyle modifications, such as dietary adjustments to avoid aggravating foods are beneficial and recommended by the Prior Art. Prior Art also recommends emotional and psychological support for patients struggling with this condition.

Elmiron® (pentosan polysulfate sodium PPS), is an FDA approved oral prescription for interstitial cystitis Elmiron® is a registered trademark of IVAX Research, Inc., presently under license to Ortho-McNeil Pharmaceutical, Inc. It has become the foundation for treatment of this condition. It has been shown to be effective in only 30% of cases PPS is a heparin-like product that is structurally similar to GAG and may help repair and restore the integrity of the damaged mucous layer. Standard dosing is 100 mg three times daily one hour before or two hours after meals. The approximate cost of Elmiron® to the patient is over $3,000.00 per year.

Another FDA approved therapy is intravesical instillation of dimethyl sulfoxide (DMSO). DMSO helps relieve pain and inflammation when administered into the bladder. Histamine released by mast cells also contributes to interstitial cystitis and DMSO may stabilize these cells It also exhibits muscle relaxant properties that can help with symptoms of urgency and frequency. Typical dosing is instillation of 50 mL DMSO (50% aqueous solution) directly into the bladder via a catheter, where it remains for 15 minutes. This is repeated every 2 weeks until symptomatic relief is achieved (usually 4-8 treatments). Maintenance dosing is usually every 1 or 2 months as needed. Administration of DMSO is usually done in a clinician's office but some patients may learn to self-catheterize at home.

There are also many other oral medications used in the treatment of the symptoms of interstitial cystitis, although not specifically FDA approved for this indication. Low dose tricyclic antidepressants such as amitriptyline, doxepin, and imipramine are beneficial in relieving pain and in decreasing urinary symptoms. Antihistamines such as hydroxyzine can help diminish the effects of histamine on the cascade of chemical mediators released upon injury. Traditional analgesics may help relieve mild discomfort, but they may also lead to the release of histamine, contributing to flare ups. Other oral medications that may be considered include neuroleptic medications such as gabapentin, urinary analgesics such as phenazopyridine, anticholinergics such as oxybutinin chloride, skeletal muscle relaxants such as cyclobenzaprine, and calcium channel blockers such as nifedipine. Intravesical instillation of heparin may also be considered for its anti-inflammatory effects, protective effects, and ability to inhibit scarring.

In addition to conventional medications for interstitial cystitis, there are various natural products available that may help control symptoms. Methylsulfonylmethane (MSM), which is also known as dimethyl sulfone (DMSO₂) has been used in practice as an alternative to DMSO. It can be administered intravesicularly, orally, topically, or intravenously. Often, a combination of these routes is used MSM does not have the same garlicky odor as DMSO so it may be better tolerated, but it may also take longer for patients to experience improved symptoms. Another approach targets nitric oxide deficit observed in interstitial cystitis patients and the symptom improvement associated with elevation of nitric oxide levels. Arginine is a natural precursor of nitric oxide and may be considered for treatment. Results from small trials have showed mixed results, but the trials may have been too small to detect statistical significance between different outcomes. Plant sterols can decrease inflammation through modulation of the immune response. Quercetin is a bioflavanoid found in red wine, onions, and green tea that reduces mast cell release of histamine and may relieve interstitial cystitis symptoms. Another promising supplement that is being studied is melatonin, which may stabilize and protect the urothelium—safety and efficacy must be established.

The treatments of the Prior Art have been found to be only marginally effective and are usually prescribed as individual treatment, though there has recently been indications of multi-modality treatments using more than one drug. However, to date the Prior Art provides no combination treatment that is widely effective in treating or causing remission of interstitial cystitis.

### SUMMARY OF THE INVENTION

The present invention relates to a formulation and method of using the formulation for the treatment of interstitial cystitis and other similar conditions of the bladder. The formulation of this invention is a highly specialized, compounded pharmaceutical that contains a combination of numerous therapeutically different medications in a unique delivery vehicle and system. The individual components of the formulation work synergistically to restore and repair each of the different issues associated with interstitial cystitis. The individual components of the formulation exist in an aqueous vehicle to facilitate drug contact with the bladder wall. The formulation is instilled directly into the bladder, commonly in a physician's office. The combination of the specific individual components in the formulation of this invention offers a benefit over the single component formulations of the Prior Art. The combination of specific individual components as disclosed is a widely effective treatment, wherein the individual components work synergistically to cure or initiate a prolonged remission of interstitial cystitis, rather than merely treat the symptoms.

The general objective of the present invention is to provide a formulation effective in treating interstitial cystitis and other similar bladder conditions.

A further objective of the present invention is to provide a method for the effective treatment of interstitial cystitis by use of a formulation.

Another objective of the present invention is to provide a formulation for the effective treatment of interstitial cystitis that provides for a novel combination of components that work synergistically in a delivery vehicle and system.

These objectives and advantages are obtained by providing a formulation and method of using the formulation to treat interstitial cystitis and other related bladder conditions as described herein. These and other objects of the present invention will become more readily apparent from a reading of the following detailed description taken in conjunction with the accompanying drawings wherein like reference numerals indicate similar parts, and with further reference to the appended claims.

### BRIEF DESCRIPTION OF THE DRAWINGS

The preferred embodiment of the invention, illustrative of the best mode in which applicant has contemplated applying the principles is set forth in the following description and is shown in the drawings and is particularly and distinctly pointed out and set forth in the appended claims. The invention may take physical form in certain parts and arrangements of parts, numerous embodiments of which will be described in detail in the specification and illustrated in the accompanying drawings which form a part hereof, and wherein:

FIG. 1 is a diagram showing the individual components of a certain embodiment of the formulation of this invention;

FIG. 2 is a diagram showing the individual components of another embodiment of the formulation of this invention;

FIG. 3 is a perspective view of the syringe and catheter used to administer certain embodiments of the formulation of the invention; and

FIG. 4 is a diagram showing the application of the formulation of a certain embodiment of the invention into the patient's bladder during administration of the formulation.

### DETAILED DESCRIPTION OF THE INVENTION

Referring now to the drawings wherein the showings are for purposes of illustrating numerous embodiments of the invention only and not for purposes of limiting the same, the figures illustrate the novel idea of a formulation and method of using the formulation for the treatment of interstitial cystitis and other similar conditions of the bladder. The formulation of this invention is a highly specialized, compounded pharmaceutical that contains four different medications in a delivery vehicle and system. The individual components of the formulation work synergistically to restore and repair each of the different issues associated with interstitial cystitis.

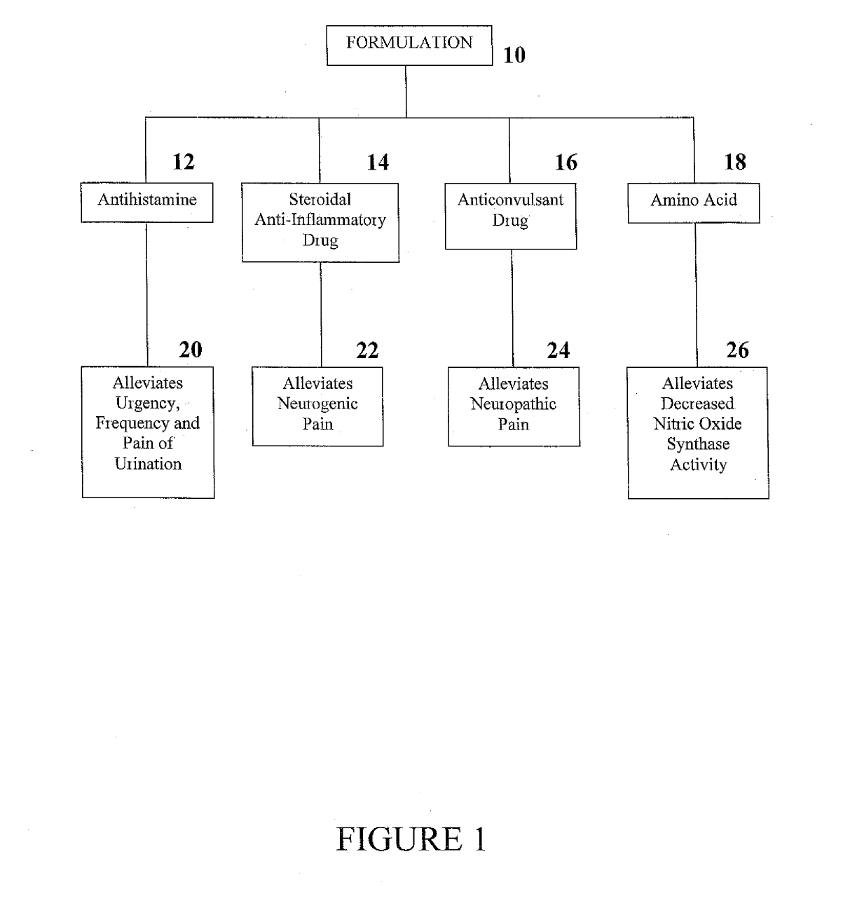

The formulation of this invention is an intravesical solution that combines individual components to provide relief of pain, urgency, and frequency associated with interstitial cystitis. As shown in the diagram of FIG. 1, the combination of individual components 12, 14, 16 and 18 of formulation 10 treats multiple symptoms 20, 22, 24 and 26 of interstitial cystitis and has the advantage of utilizing different mechanisms of action to ameliorate the symptoms of interstitial cystitis.

Individual component 12 of formulation 10 of this invention is an antihistamine agent. A preferred embodiment of the invention uses the antihistamine Hydroxyzine, however it is foreseen by this invention that similar antihistamines may be used within the formulation and accomplish the same outcome. The chemical composition of the preferred antihistamine, hydroxyzine is shown below:

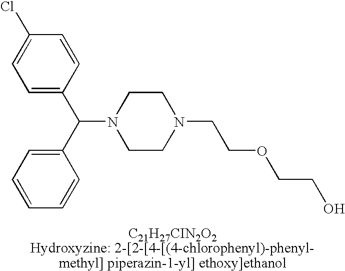

Non-limiting examples of further antihistamines that may be effective within the formulation at hand are: aminoalkyl ethers (including but not limited to bromazine, carbinoxamine, clemastine, chlorphenoxamine, diphenylpyraline, diphenhydramine, and doxylamine); substituted alkylamines (including but not limited to brompheniramine, chlorphenamine, dexbrompheniramine, dexchlorpheniramine, dimetindene, pheniramine, and talastine); substituted ethylene diamines (including but not limited to chloropyramine, histapyrodine, mepyramine, methapyrilene, and pyribenzamine); phenothiazine derivatives (including but not limited to alimemazine, hydroxyethylpromethazine, isothipendyl, mequitazine, methidilazine, oxomemazine, and promethazine); and piperazine derivatives (including but not limited to buclizine, cetrizine, chlorcyclizine, cinnarizine, cyclizine, hydroxyzine, levocetrizine, meclozine, niaprazine, and oxatomide).

The chemical formulas for a number of these further non-limiting examples of antihistamines are shown below:

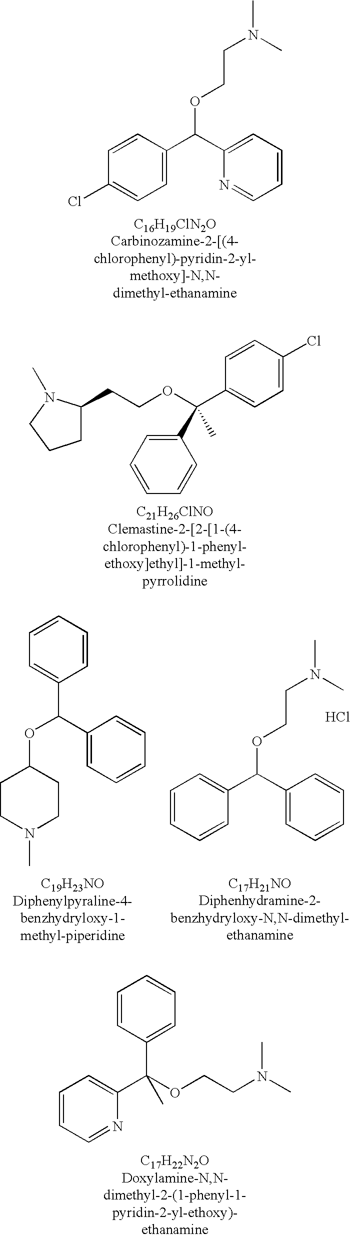

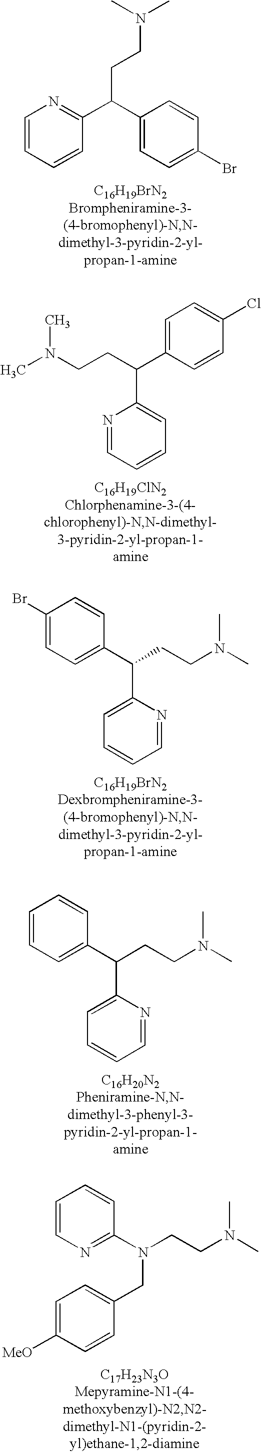

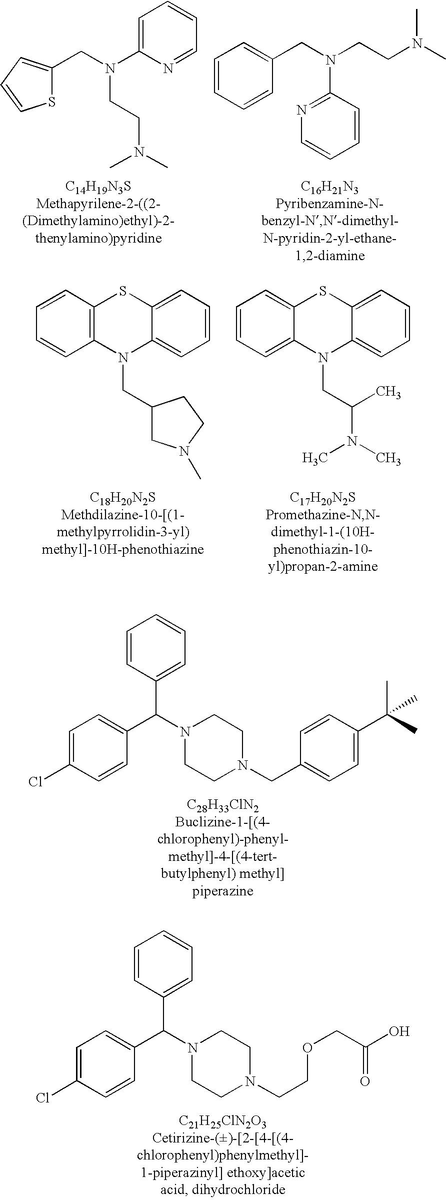

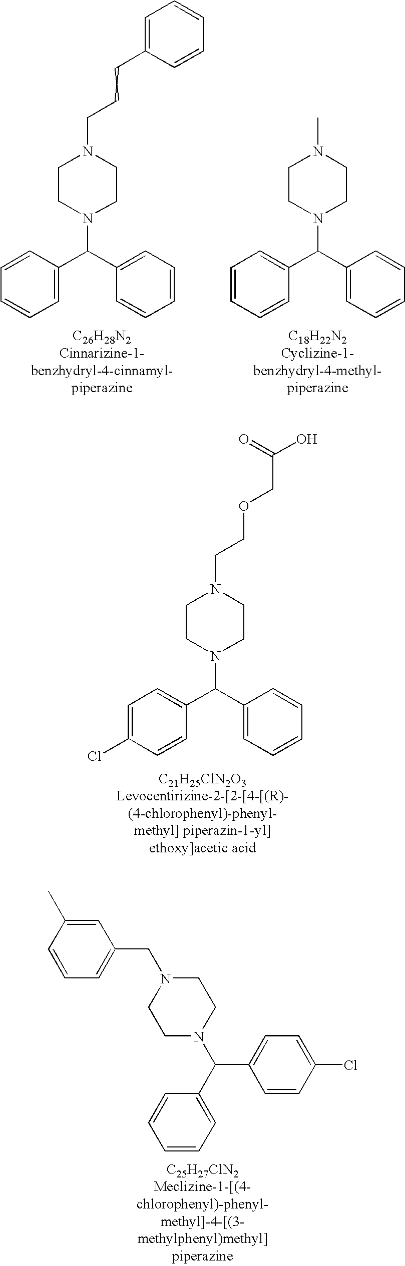

Antihistamines are commonly prescribed to manage allergic reactions by blocking histamine from binding to the Histamine₁-receptor which would otherwise cause allergic symptoms. An antihistamine is advantageous within formulation 10 of this invention because the bladders of patients with interstitial cystitis have a higher than normal concentration of the cells that release histamine. Antihistamines, including Hydroxyzine, also have anticholinergic and analgesic properties which are advantageous in decreasing symptoms 20 of interstitial cystitis, namely the urgency, frequency, and pain of urination associated with interstitial cystitis.

Another individual component 14 of formulation 10 includes the use of a steroidal anti-inflammatory agent or drug A preferred embodiment of the invention uses the steroidal anti-inflammatory drug Dexamethasone, however it is foreseen by this invention that similar steroidal anti-inflammatory drugs may be used within the formulation and accomplish the same outcome. The chemical composition of the preferred steroidal anti-inflammatory drug, Dexamethasone is shown below:

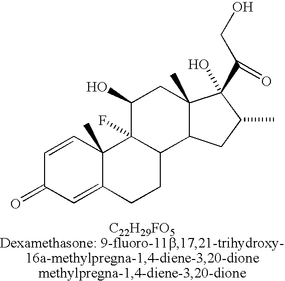

Non-limiting examples of further steroidal anti-inflammatory drugs that may be effective within the formulation at hand are: corticosteroids, glucocorticoid/recetors and mineralocorticoid/receptors, including but not limited to alclometasone, aldosterone, amcinonide, beclometasone, betamethasone, budesonide, ciclesonide, clobetasol, clobetasone, clocortolone, cloprednol, cortisone, cortivazol, deflazacort, deoxycorticosterone, desonide, desoximetasone, desoxycortone, dexamethasone, diflorasone, diflucortolone, difluprednate, fluclorolone, fludrocortisones, fludroxycortide, flumetasone, flunisolide, fluocinolone, fluocinonide, fluocortin, fluocortolone, fluorometholone, fluperolone, fluprednidene, fluticasone, formocortal, halcinonide, halometasone, hydrocortisonelcortisol, loteprednol, medrysone, meprednisone, methylprednisoline, methylprednisolone, mometasone, paramethasone, prednicarbate, prednisone, prednisolone, prednylidene, rimexolone, tixcortol, triamcinolone, and ulobetasol and their associated salts and esters.

The chemical formulas for many of the non-limiting examples are shown below:

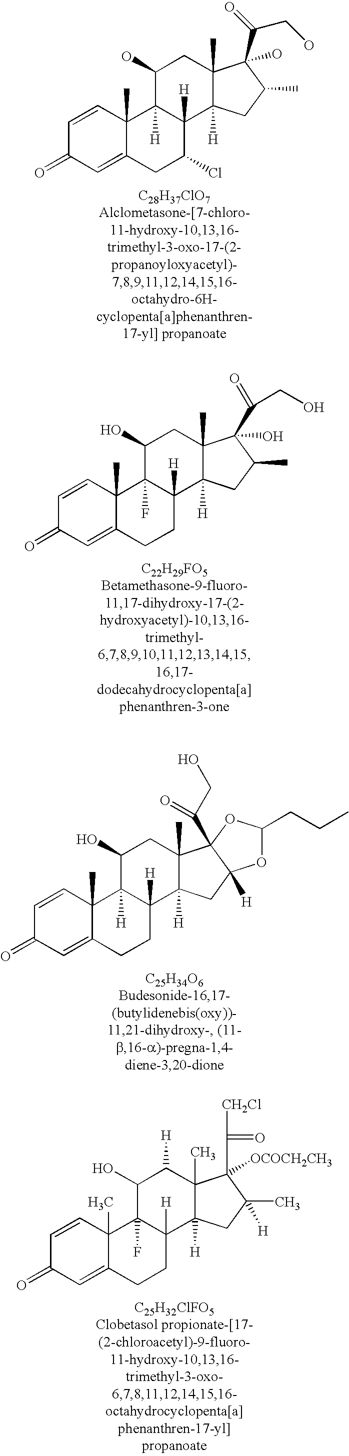

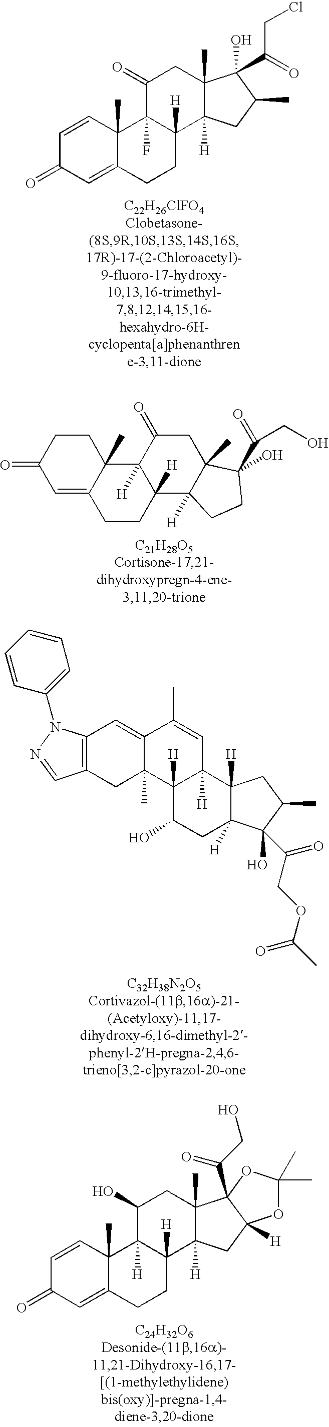

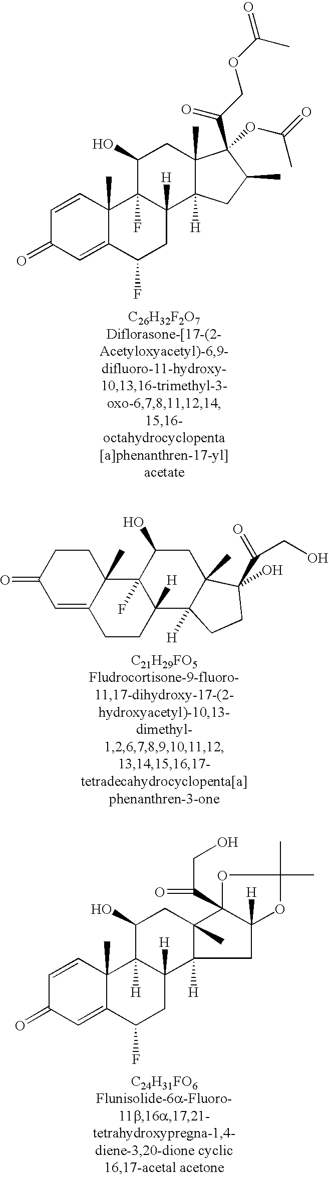

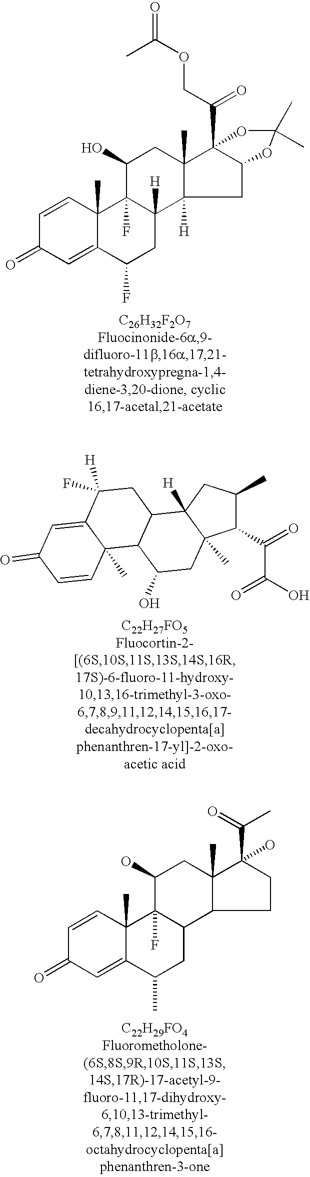

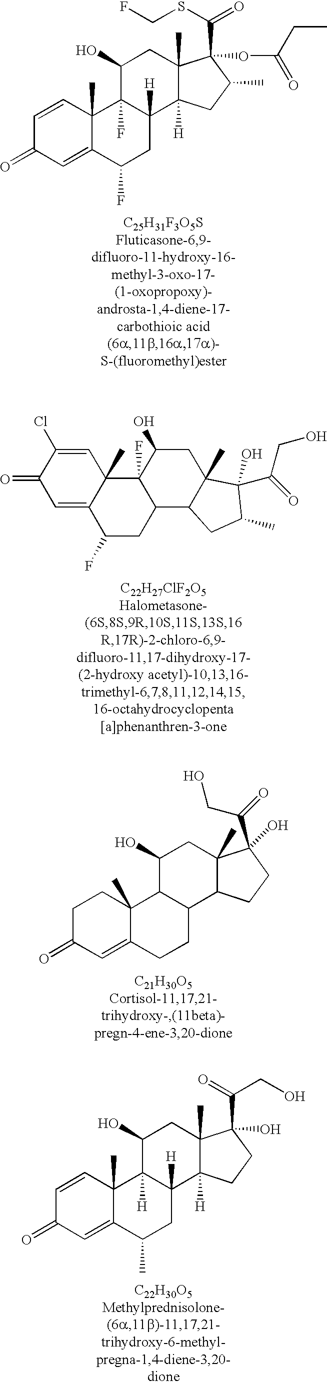

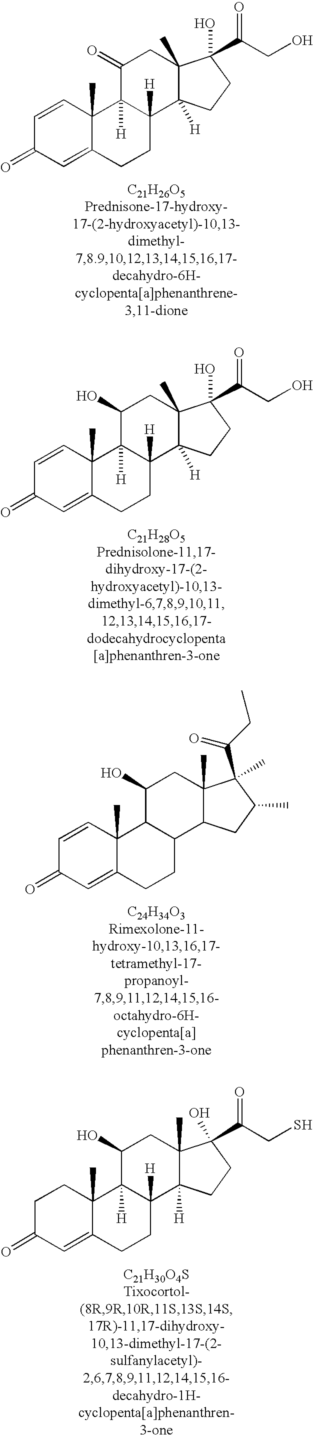

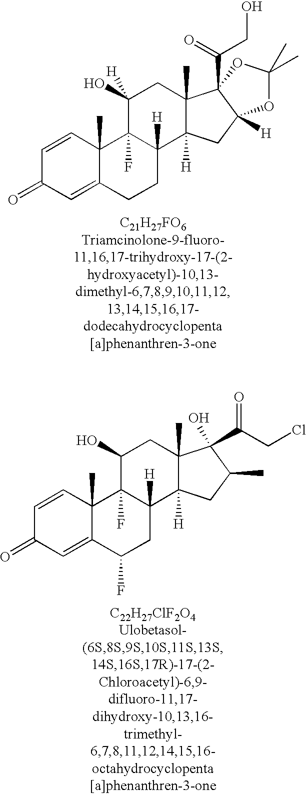

The steroidal anti-inflammatory agent or drug inhibits the production of inflammatory mediators and suppresses the immune system. The steroidal anti-inflammatory drug is beneficial to formulation 10 of this invention because interstitial cystitis is associated with hyperactive immune responses and neurogenic pain. The steroidal anti-inflammatory drug within formulation 10 resolves symptom 22 alleviating the neurogenic pain.

Yet another individual component 16 of formulation 10 includes the use of an anticonvulsant agent or drug. Preferred embodiments of the formulation use an anticonvulsant drug synthesized to mimic the structure of GABA, whose chemical formula can be seen below:

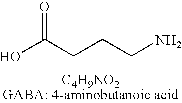

A preferred embodiment of the invention uses the anticonvulsant drug Gabapentin, however it is foreseen by this invention that similar anticonvulsant drugs may be used within the formulation and accomplish the same goals. The chemical composition of the preferred anticonvulsant drug Gabapentin can be seen below:

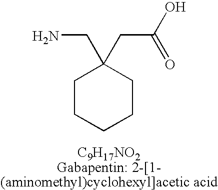

Non-limiting examples of further anticonvulsant drugs that may be effective within the formulation at hand are: barbiturates, hydantoins, oxazolidinediones, succinimides, benzodiazepines, carboxamides, fatty acid derivatives, carboxylic acids, and GABA analogs (including but not limited to baclofen, gabapentin, pregabalin, progabide, and vigabatrin).

The chemical formulas for many of the non-limiting examples are shown below:

| | |
|---|---|
| 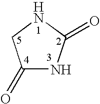 | 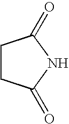 |
| C₃H₄N₂O₂ | C₄H₅NO₂ |
| Hydantoins - imidazolidine- | Succlinimide - Pyrrolidine- |
| 2,4-dione | 2,5-dione |

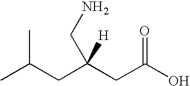

C₈H₁₇NO₂
Pregabalin - (S)-3-
(aminomethyl)-5-
methylhexanoic acid

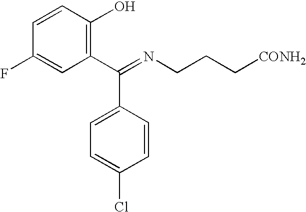

C₁₇H₁₆ClFN₂O₂
Progabide - 4-[(4-
chlorophenyl)-(5-fluoro-2-
hydroxy-phenyl)-
methylidene]
aminobutanamide

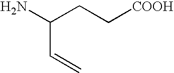

C₆H₁₁NO₂
Vigabatrin - 4-aminohex-
5-enoic acid

The anticonvulsant drug affects the nerve tissue and effectively treats neuropathic pain. The anticonvulsant drug is advantageous to formulation 10 of this invention by treating symptoms 24, namely neuropathic pain that is caused by an increase in endogenous pain-transmitting substances in interstitial cystitis patients.

Formulation 10 of this invention also includes individual component 18 of an amino acid that is the precursor in the production of nitric oxide, a vasodilator, which is naturally made in the body by the enzyme nitric oxide synthase. The chemical composition of the amino acid used in the formulation of this invention is any molecule that contains both amine and carboxyl functional groups A preferred embodiment uses the amino acid Arginine, however it is foreseen by this invention that similar amino acids may be used in the formulation and accomplish the same outcomes. The chemical composition of the preferred amino acid Arginine can be seen below:

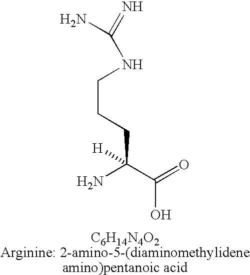

Non-limiting examples of further amino acids include alanine, asparagine, aspartic acid, cysteine, glutamic acid, glutamine, glycine, histidine, isoleucine, leucine, lysine, methionine, phenylalanine, praline, serine, threonine, tryptophan, tyrosine, and valine.

The chemical formulas for many of the non-limiting examples are shown below:

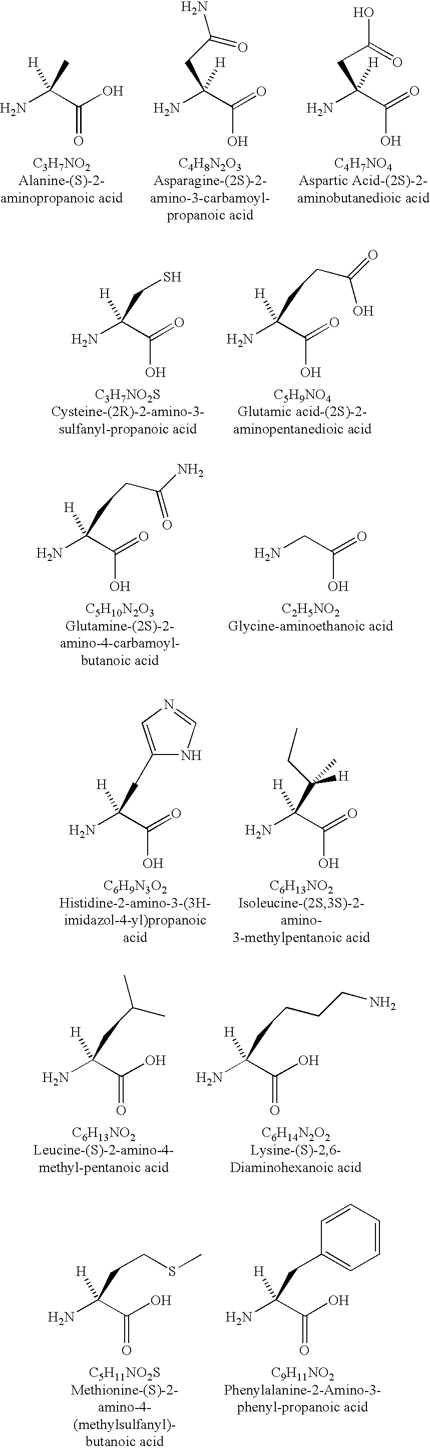

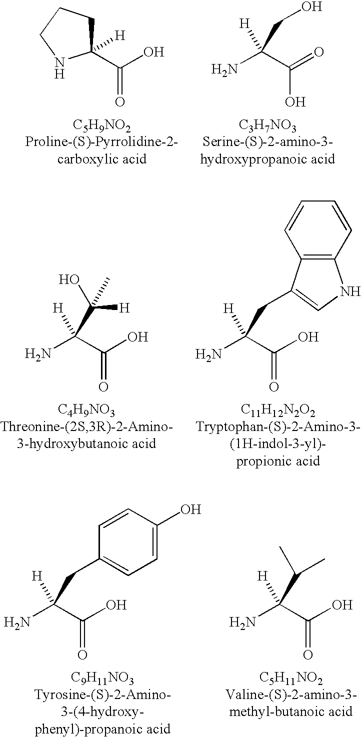

The use of such an amino acid increases nitric oxide synthase activity, which is beneficial to formulation 10 of this invention because interstitial cystitis patients have notably decreased nitric oxide synthase activity within their bladders. Supplementing the natural substrate of the amino acid enzyme within the bladder helps to repair the bladder wall and eliminates symptom 26, namely the decreased nitric oxide synthase activity, one of the factors that is believed to cause interstitial cystitis.

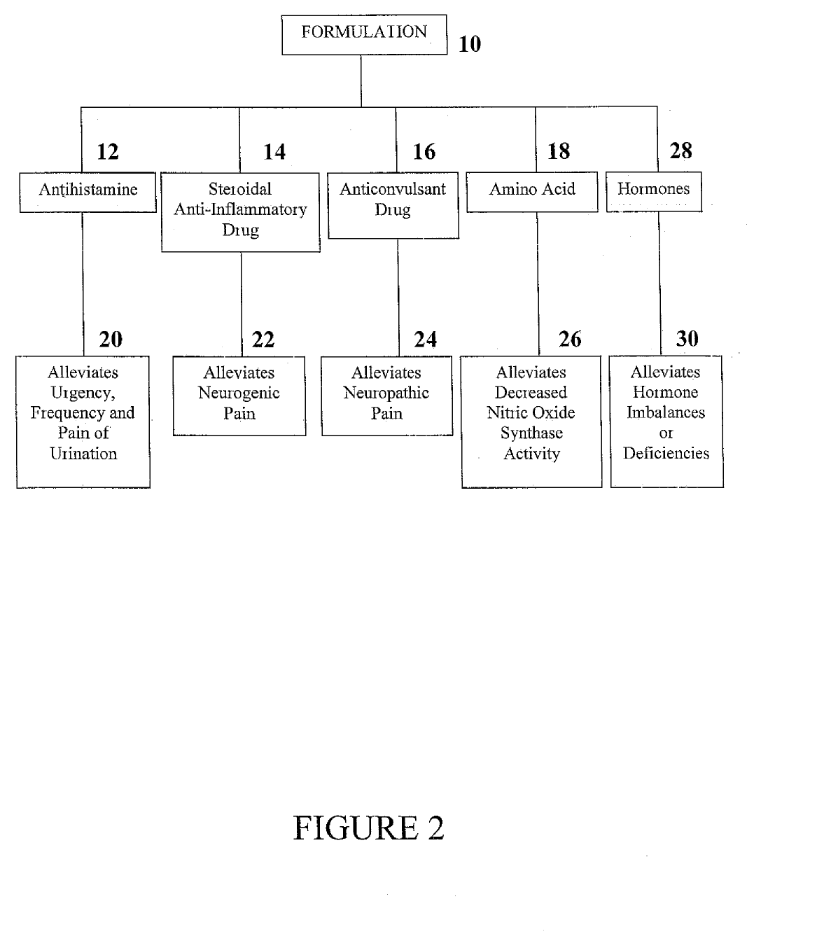

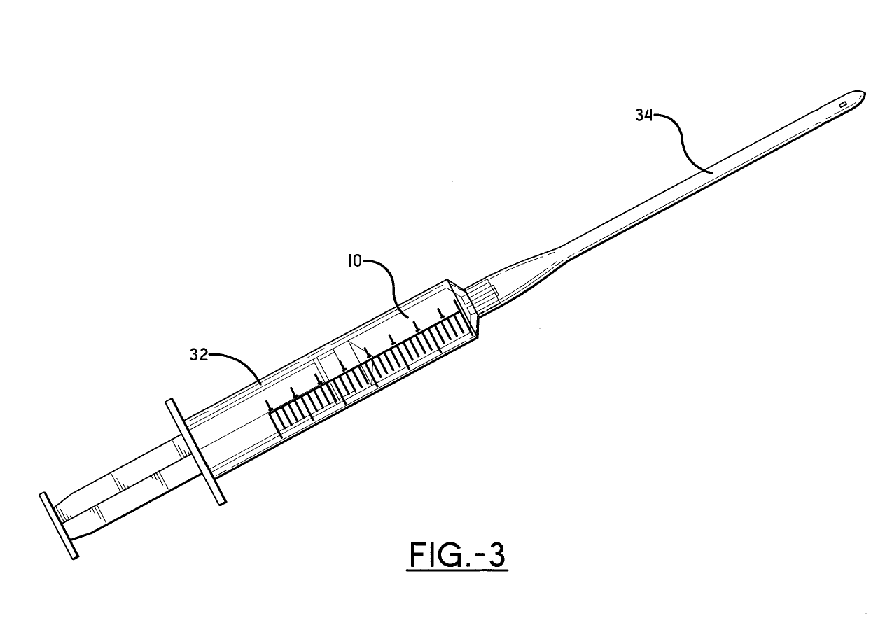

Further embodiments of formulation 10 of this invention, as shown in FIG. 2, may include various hormone supplements 28, in addition to four individual components 12, 14, 16 and 18 of formulation 10. The majority of interstitial cystitis patients are females, and an underlying hormone imbalance is often noted. Bio-identical hormone replacement therapy may be combined with the interstitial cystitis treatment formulation of this invention, with the hormones administered by a separate formulation administered concurrently with the formulation of this invention (not shown), or within the same formulation, as shown in FIG. 3. Component 28 of hormone supplements are beneficial to formulation 10 by relieving symptom 30 of an underlying hormone imbalance or deficiency. The types and levels of the hormone components of the formulation of this invention may be determined by the physician on a patient by patient basis.

As is known within the art, components 12, 14, 16 and 18, and in some embodiments additional component 28, are combined within the formulation in combination with a liquid filler solution. The liquid filler solution may, but does not necessarily include solubilizing agents, for the purpose of solubilizing the components of the formulation into a liquid formulation of a larger volume than the combination of the components alone. The filler liquids may be used to increase the volume of the formulation to allow the formulation to have a sufficient volume to contact larger areas of the patient's bladder. The filler liquids may also be used to increase the flow rates and pressure within the syringe to allow the formulation to be instilled into the patients bladder in a more efficient fashion. The total volume or amount of the formulation of this invention is necessarily limited only by the patient's bladder capacity. In certain preferred embodiments the formulation as administered to patients may have a volume of approximately 30-60 mL. It is foreseen, however, that the overall volume of the formulation may, however, be less than or greater than 30-60 mL, without affecting the performance or purpose of the invention.

The components of the formulation of this invention may be combined in equal or unequal parts and may be combined by set weights, volumes or percentages by weight or volume. A minimum amount of each component must be present within the formulation such that the component is effective to synergistically treat the condition when combined with the other components. In preferred embodiments of the formulation components 12, 14, 16 and 18 are present within the formulation as between 0.01-20% of the overall volume of the formulation, and the remaining percentage of the formulation contains a filler solution. In certain preferred embodiments of the formulation of the present invention, antihistamine component 12 is present in the formulation as 0.25% of the overall volume of the formulation, steroidal anti-inflammatory drug component 14 is present in the formulation as 0.05% of the overall volume of the formulation, anti-convulsant component 16 is present in the formulation as 0.3% of the overall volume of the formulation, and amino acid component 18 is present in the formulation as 2.75% of the overall volume of the formulation, with filler solution comprising approximately 96.65% of the formulation. The composition of the formulation is not by any means limited to the quantities given in the previous example, and it is easily foreseen by this invention that further quantities may be combined to achieve the same purpose and objectives of this invention. For example, the formulation may consist of 1% of each component, with 96% of the formulation comprised by a filler solution. The components may be combined in any quantities such that the synergistic effects of the formulation occur for the treatment of interstitial cystitis or other related bladder conditions.

The formulation of this invention is commonly, but not necessarily, prepared by a pharmacist in a clean-room system. Clean-room systems allow the formulation to be prepared without contamination, maintaining the sterility and integrity of the formulation. The individual components of the formulation exist in an aqueous vehicle to facilitate drug contact with the bladder wall. As shown in FIG. 3, formulation 10 is commonly administered from syringe 32 using catheter 34. In preferred embodiments of the invention, the formulation is administered using a 30 mL syringe, and a lubricated latex-free catheter, however any syringe and catheter can be used to administer the drug. Administration of the formulation directly into the bladder is greatly preferable, because administration into the bladder prevents absorption of the formulation into the blood stream to any significant degree. This reduces the chance of drug interactions, adverse side effects and overdose.

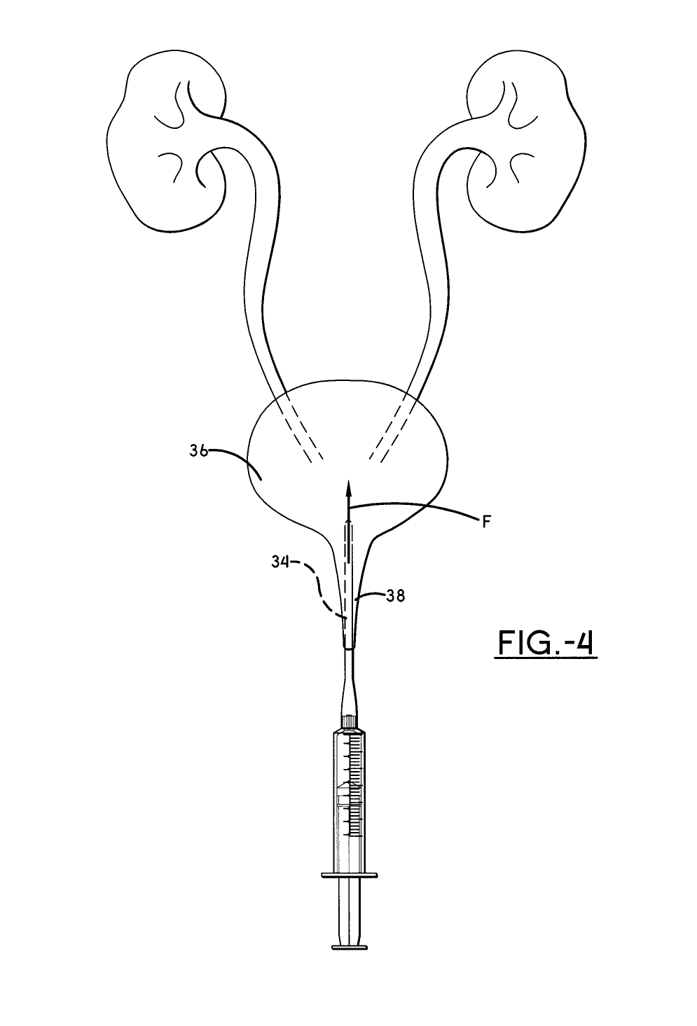

Formulation 10 is instilled directly into bladder 36, as shown in FIG. 4, and is commonly administered in a physician's office, however certain patients may be able to self-catheterize and administer the formulation themselves Catheter 34 is inserted into urethra 38, and formulation 10 is injected into bladder 36 using syringe 32 and catheter 34, as is commonly known within the art. Upon instillation through syringe 32 and catheter 34, formulation 10 flows F into bladder 36 making contact with the tissue of the walls of bladder 36.

Certain embodiments of the invention may require a single instillation of the formulation of this invention, while other embodiments may require more than one instillation of the formulation. A preferred embodiment of the invention requires instillation seven times over a two week period, commonly requiring instillation over five consecutive days, with the remaining two instillations occurring over the following nine days.

In certain embodiments of the invention no further supplemental applications may be necessary after the originally prescribed dosages of the formulation. In other embodiments a single or more supplemental applications of the formulation may be necessary at some time after the originally prescribed dosages of the formulation. The supplemental dosages may be required weeks, months, or even years after the original dosage, as necessary if a recurrence or exacerbation of the condition is noticed by the patient or health care professional treating the patient. The frequency of instillation of the originally prescribed dosage of the formulation or the subsequent supplemental dosage of the formulation may be determined by the health care professional treating the patient suffering with interstitial cystitis or the related bladder condition.

In the foregoing description, certain terms have been used for brevity, clearness, illustration and understanding; but no unnecessary limitations are to be implied therefrom beyond the requirements of the Prior Art, because such terms are used for descriptive purposes and are intended to be broadly construed. Moreover, this invention has been described in detail with reference to specific embodiments thereof, including the respective best modes for carrying out each embodiment. It shall be understood that these illustrations are by way of example and not by way of limitation.

What is claimed is:

1. A formulation for treatment of a patient having interstitial cystitis, said formulation comprising:
an antihistamine agent;
a steroidal anti-inflammatory agent;
an anticonvulsant agent; and
an amino acid;
wherein the antihistamine agent, the steroidal anti-inflammatory agent, the anticonvulsant agent, and the amino acid are combined within a liquid filler solution.

2. The formulation of claim 1 wherein the formulation further comprises a hormonal supplement.

3. The formulation of claim 1 wherein the liquid filler solution includes a solubilizing component.

4. The formulation of claim 1 wherein the antihistamine agent is Hydroxyzine.

5. The formulation of claim 1 wherein the steroidal anti-inflammatory agent is Dexamethasone.

6. The formulation of claim 1 wherein the anticonvulsant agent is Gabapentin.

7. The formulation of claim 1 wherein the amino acid is Arginine.

8. A formulation for treatment of a patient having interstitial cystitis, said formulation having a volume, and said formulation comprising:
Hydroxyzine;
Dexamethasone;
Gabapentin; and
Arginine;
wherein the Hydroxyzine, the Dexamethasone, the Gabapentin, and the Arginine are combined within a liquid filler solution.

9. The formulation of claim 8 wherein the liquid filler solution includes a solubilizing component.

10. The formulation of claim 8 wherein the formulation is composed of approximately 0.25% Hydroxyzine, approximately 0.05% Dexamethasone, approximately 0.3% Gabapentin, approximately 2.75% Arginine, and approximately 96.65% liquid filler solution.

11. A method of treating interstitial cystitis, the method comprising:
inserting a catheter into a patient's urethra;
aligning a syringe with the catheter such that a liquid pathway exists between the syringe and the catheter, wherein the syringe contains a liquid formulation, the liquid formulation comprising an antihistamine agent, a steroidal anti-inflammatory agent, an anticonvulsant agent, an amino acid, and a filler solution;
instilling the liquid formulation into the patient's bladder by injecting the liquid formulation from the syringe through the catheter into the bladder of the patient.

12. The method of claim 11 wherein the liquid formulation further comprises a hormonal supplement.

13. The method of claim 11 wherein the filler solution of the liquid formulation includes a solubilizing component.

14. The formulation of claim 11 wherein the antihistamine agent of the liquid formulation is Hydroxyzine.

15. The formulation of claim 11 wherein the steroidal anti-inflammatory agent of the liquid formulation is Dexamethasone.

16. The formulation of claim 11 wherein the anticonvulsant agent of the liquid formulation is Gabapentin.

17. The formulation of claim 11 wherein the amino acid of the liquid formulation is Arginine.
